(19) United States
(12) Patent Application Publication
(10) Pub. No.: US 2019/0392953 A1
(43) Pub. Date: Dec. 26, 2019

(54) A SYSTEM AND METHOD OF DIAGNOSIS SKIN AND TISSUE LESIONS AND ABNORMALITIES

(71) Applicant: DERMADETECT LTD., Jerusalem (IL)

(72) Inventors: Guy Mordechay STEUER, Ganei-Tikva (IL); Arieh INGBER, Petach Tikva (IL); Gennadi SCHECHTMANN, Cologne (DE); Eugene DIKER, Jerusalem (IL)

(21) Appl. No.: 16/485,184

(22) PCT Filed: Feb. 11, 2018
(86) PCT No.: PCT/IL2018/050159

§ 371 Date: Aug. 12, 2019

Related U.S. Application Data
(60) Provisional application No. 62/577,043, filed on Oct. 25, 2017.
(60) Provisional application No. 62/457,861, filed on Feb. 11, 2017.

(51) Int. Cl. G16H 50/20; G16H 20/10; G16H 30/20

(52) U.S. Cl. CPC G16H 50/20; G16H 30/20; G16H 20/10

(57) ABSTRACT

A computerized system and method for skin lesion diagnosis. The user enters one or more images of the lesion and answers an online questionnaire. Using image analysis techniques, information from the questionnaire, information from the user profile and optionally additional external information, the system makes a diagnosis of the lesion.

### TECHNICAL FIELD

The present invention relates to a system and method for diagnosing skin and tissue lesions and abnormalities in general, and, in particular, to a computerized system and method for diagnosing skin and tissue lesions and abnormalities.

### BACKGROUND ART

Dermatology is a spot diagnosis field of medicine in which skilled dermatologists are capable of intuitive determination/conjecture of the correct diagnosis of lesions, such as skin-lesions, using pre-learned characteristic lesion properties as well as previous experience to narrow differential diagnosis of a skin lesion to few reasonable options. Thus, correct diagnosis significantly depends on individual professional knowledge and previous experience and is hampered by typical human subjective bias as well as limited experience, fatigue, stress etc.

On the other hand, telemedicine shows that many the diagnosis of skin lesions can be made by a spot diagnosis.

The term “skin lesion” and its derivatives as used herein refer to epidermal abnormalities and other issues of body tissue that are imaginable tissues, such as eye/iris/eyelash/sclera, genitalia, nails, hair, mucosa and so on.

The term “rushes” as used herein refer to group of skin lesions which together make a specific diagnosis or disease.

There are a relatively small number of skin lesion types and rashes, and a specific skin lesion can be defined by either a predefined number of parameters and/or visual characteristics. There are numerous databases holding millions of examples of skin lesions. There is thus a need in the industry to provide a system and method to take advantage of these databases and allow visual imaging processing by machine learning or deep learning techniques via automatic visual processing techniques to diagnose skin lesions.

### SUMMARY OF INVENTION

It is an object of the present invention to provide a system and method for diagnosing skin lesions.

It is another object of the present invention to provide skin/tissue lesion and abnormality recognition.

It is a further object of the present invention to provide a system and method for diagnosing and managing acute skin lesions of patients from the time of appearance until a dermatologist/physician clinic visit.

It is yet a further object of the present invention to provide a system and method for diagnosing skin lesions to enhance the dermatologist/physician accuracy of diagnosis.

It is yet another object of the present invention to provide a system and method for diagnosing skin lesions to serve as an aid/tool for other physicians, medical students, pharmacists, cosmetic professionals and patients.

It is yet another object of the present invention to provide a system and method for diagnosing skin lesions and skin problems to serve as an aid/tool and adapting the interface for other physicians, medical students, pharmacists, cosmetic professionals and patients.

It is yet another object of the present invention to provide a system and method for diagnosing skin lesions from patients/humans but also from textbooks/atlas/photos.

It is yet another object of the present invention to provide a system and method for recommending treatment for the diagnosed skin lesions and skin problems to serve as an aid/tool for other physicians, medical students, pharmacists, cosmetic professionals and patients, for diagnosed acute, chronic, congenital, drug related (side effects) skin lesions and problems (e.g. such as pruritus).

It is yet another object of the present invention to provide a system and method for managing skin lesions and problems (e.g. hair loss, dry/oily skin) to serve as an aid/tool for other physicians, medical students, pharmacists, cosmetic professionals and patients, for diagnosed acute, chronic, congenital, drug related (side effects) skin lesions and problems.

It is yet another object of the present invention to provide a system and method for offering an efficient use for a specific medicament to serve as an aid/tool for other physicians, medical students, pharmacists, cosmetic professionals and patients, for diagnosed acute, chronic, congenital, drug related (side effects) skin lesions and problems (e.g. such as pruritus). The prescribed non-OTC drugs may vary from country regulations.

It is yet another object of the present invention to provide a system and method for recommending and/or prescribing a medicament for a specific disease to serve as an aid/tool for other physicians, medical students, pharmacists, cosmetic professionals and patients.

It is yet another object of the present invention to provide a system and method for prescribing an OTC (Over the Counter) drug to serve as an aid/tool for other physicians, medical students, pharmacists, cosmetic professionals and patients. It is yet another object of the present invention to provide a system and method for prescribing a non-OTC (such as antibiotic) drug to serve as an aid/tool for other physicians, medical students, pharmacists, cosmetic professionals and patients.

It is yet another object of the present invention to provide a system and method for follow up (e.g. such as reminding taking photos of the condition overtime and/or in a specific time, for better diagnosing of a disease that typically change such as varicella or cellulitis or to look after lesion that can become inflamed such as psoriasis or malignant overtime such as actinic keratosis) to serve as an aid/tool for other physicians, medical students, pharmacists, cosmetic professionals and patients.

It is yet another object of the present invention to provide a system and method for connecting and/or scheduling an appointment with the nearby available professional (based on your GEO location) to serve as an aid/tool for other physicians, medical students, pharmacists, cosmetic professionals and patients.

It is yet another object of the present invention to provide a system and method for listing nearby pharmacies based on your GEO location related to the treatment needed (e.g. “the drug that you need is located in location X (CVS for example) and costs $69 and in location Y (such as Walgreen) it costs $73), to serve as an aid/tool for other physicians, medical students, pharmacists, cosmetic professionals and patients.

It is yet another object of the present invention to provide a system and method for generating professional anamnesis by collecting data received from the patient (e.g. history, pictures and lab results) by using a chatbot, guided questionnaire and/or any related information about the condition, to serve as an aid/tool for other physicians, medical students, pharmacists, cosmetic professionals and patients.

It is yet another object of the present invention to provide a system and method for merging/combining information received from the patient by method of questioning AND information received by analyzing a photo of a condition/lesion, to serve as an aid/tool for other physicians, medical students, pharmacists, cosmetic professionals and patients.

It is yet another object of the present invention to provide a system and method for offering medicament based on authorized medication covered by HMO's or the medical insurance, to serve as an aid/tool for other physicians, medical students, pharmacists, cosmetic professionals and patients.

The present invention relates to a computer system for diagnosing skin lesions of a user, comprising: a technology (mobile/web/API/SDK), which receives as an input an image and/or video, and outputs a diagnosis and/or treatment pathway for the user. This technology will use the information received from the user (patient), by means of guided dynamically changed questionnaire, and also information extracted from analysis of one or more lesion images. Both types of information (questionnaire responses and image analysis) are combined, in order to improve the accuracy of the diagnosis (as opposed to only using the information extracted from the image by means of computer vision techniques). The system comprises a processor; and a memory communicatively coupled to the processor comprising computer-readable instructions that when executed, by the processor cause the computer system to:

i. acquire via the processor information from the user by means of questionnaire(s)/chatbot:
a. each question to the user affects the following questions in terms of content and quantity;
b. the answer of the user affects the analysis engine and/or affects the next presented question;
   ii. acquire at least one lesion image;
   iii. apply via the processor analysis of the image so to extract features (such as lesion color, elevation, shape, etc.);
   iv. present the user via the processor a first question about the lesion;
   v. acquire via the processor the user's response to said first question;
   vi. process via the processor the user's response, user lesion image and user profile and if a diagnosis cannot be made, decide whether to formulate another question to the user or request an additional image (such as a close-up or an image from another distance, or with different light conditions) of the lesion;
   vii. repeat steps ii to vi until a diagnosis can be made; and
   viii. present via the processor the diagnosis to the user via a textual/graphical interface such as a mobile phone screen, monitor, etc.

Optionally one or both of the following can be implemented:

ix. the diagnosis can be presented with an estimated percentage of accuracy;
x. the diagnosis can be presented together with differential diagnosis by likelihood.

In some embodiments, information about the user profile such as age, gender, existing medical conditions etc. can be obtained automatically by the system instead of questioning the user, for example, by accessing a database.

The system comprises a mobile computing device, wherein the mobile computing device includes a digital camera configured to acquire the patient lesion image, and wherein the mobile computing device is further configured with an application for acquiring the patient data.

In some embodiments, the questions are formulated, and the responses processed by a chatbot engine based on artificial intelligence approaches. The chatbot engine improves the quality of questions (in terms of asking specific questions, based on presented conditions or information from database) and quality of diagnosis the more data it processes (using machine/deep learning approaches).

In some embodiments, the system recommends a treatment along with the diagnosis.

In some embodiments, the diagnosis is provided with a strength (e.g. the differential diagnoses are presented in percentile of likelihood) relating to the certainty of the diagnosis. The likelihood of diagnosis may or may not be shown to the patient.

In some embodiments, the system can set appointments or offer contact information to geographically close located physicians, cosmetic personnel, pharmacies or hospitals.

In another aspect, the present invention relates to a reference card (in order to improve the analysis of the image features), comprising: at least one inner opening and/or a transparent field; a ruler; and a color scale, such that when a user places the inner opening and/or a transparent field above (over) a skin lesion and takes a photograph of the lesion, the ruler and color scale help determine the actual color and size of the lesion by calibration to a known ruler/scale of colors. The Region of interest may be determined by either the reference card or by the user while using the app/software tool

Lesion images can be analyzed by computer vision approaches (including latest deep learning techniques, using CNN: convolutional neural networks approaches) and then categorized into, characteristics (such as shape, contour, size, texture, color, etc). Every extracted feature from the image can be used as a category.

Lesions can be categorized into primary lesions (such as pruritic rash, macule, papule, patch, plaque, vesicle, bulla, nodule, tumor, wheal, and pustule. Secondary lesions are a modification of primary lesions) or secondary lesions, such as scratch secondarily to the pruritus the primary lesion induced, or an injury to the skin (including deformations e.g. of the fingers such as in scleroderma.

In another aspect, the present invention relates to a computer system for dermatological side effects of a drug administered to a patient, comprising: a technology (mobile/web/API/SDK), which receives as an input an image and/or video and/or drug description, and outputs a management plan for dermatological side effects for the user. The system uses the information received from the user, by means of guided dynamically changed questionnaire, information about the administered drug and also information extracted from analysis of the lesion image. The system comprises: a processor; and a memory communicatively coupled to the processor comprising computer-readable instructions that when executed by the processor cause the computer system to:

i. acquire information from the user by means of questionnaire/chatbot, and/or acquire information about the administered drug, by means of taking a photo of the drug package and/or entering directly the information about the drug by the user, wherein
a. each question to the user affects the rest of the questions and in terms of content and quantity (of questions);
b. the answer of the user affects the analysis engine and/or affects the next presented question;
   ii. acquire at least one user lesion image;
   iii. apply analysis of the image so to extract features (such as lesion color, elevation, shape, etc.) via the processor one or more pattern recognition or deep learning techniques on the acquired image to identify a correlated known lesion type;
   iv. present the user via the processor a first question about the lesion;
   v. acquire via the processor the user's response to said first question;
   vi. process via the processor the user's response, user lesion image and user profile and if a diagnostic cannot be made, decide whether to formulate another question to the user or request an additional image of the lesion;
   vii. repeat steps ii to vi until a diagnostic can be made; and
   viii. present via the processor the diagnosis to the user textual/graphical interface, such as mobile phone screen, monitor, etc.

Optionally (perform one or more of):

ix. the diagnosis can be presented with an estimated percentage of accuracy;
x. the diagnosis can be presented together with differential diagnosis by likelihood;
xii. Inform the user whether the skin lesion is related or not to the administered drug.

In some embodiments, the system recommends an appropriate management and/or treatment if the skin lesion is identified to be related to the administered drug.

The present invention focuses on the major, dangerous and important side effect of systemic and topical drugs used in dermatology and other medical fields. Suggestions for laboratory monitoring, information on record keeping, patient surveillance, and patient education from a nursing and physician perspective may be given. Since the majority of major side effects are reversible and easily managed at an early stage but might be dangerous if left untreated a careful laboratory, clinical, pictorial monitoring and follow up might be used.

An example of such administered drug are acne medications such as isotretinoin, a drug that may have many side effect among them very common and relatively benign and treatable such as dryness and irritation and some are dangerous and relatively rare such as SAPHO or a drug that has many potentially life threatening side effect such as many chemotherapeutic agents (e.g. Vectibix-Panitumumab), which has dermatologic side effects such as Acneiform eruption (papulopustular rash in 32-57%), skin fissures, paronychia, angioedema, photosensitivity, generalized exfoliative dermatitis (9-18%) and some are life-threatening such as Skin necrosis, necrotizing fascitis and abscesses, some of them warrant immediate reaction that without an immediate automatic diagnosis might be dangerously delayed to the physician visit.

In another aspect, the system acquires drug picture and/or name and informs the inquirer what are the common indications for using that drug. For example, if the inquirer shows a picture or types a name of an antifungal drug the system can inform of the typical usage as well as common regiment, proper drug usage (e.g. apply on affected skin after cleansing with soap, 2 times per day), and common side effects.

### BRIEF DESCRIPTION OF DRAWINGS

FIG. 1 is a flowchart of an embodiment of a skin lesion diagnosis method according to the present invention.

FIGS. 2-10 are illustrative views of a device-based application exemplary screenshots for skin lesion recognition, according to an embodiment of the present invention.

FIG. 11 shows a reference card, according to one embodiment of the present invention.

### DETAILED DESCRIPTION OF THE INVENTION

In the following detailed description of various embodiments, reference is made to the accompanying drawings that form a part thereof, and in which are shown by way of illustration specific embodiments in which the invention may be practiced. It is understood that other embodiments may be utilized, and structural changes may be made without departing from the scope of the present invention.

In one mode or aspect, embodiments of the invention are intended as a tool for use by a healthcare provider, who could be a general-practice physician, a plastic surgeon or a lay cosmetic practitioner. The invention can also be used by dermatologists to improve their diagnoses and personal knowledge base. The health care provider can be the one performing the imaging/video of a patient and/or can also help regarding filling out questionnaires.

Regardless of any interaction with a health care provider, the invention is configured to provide information to help with the identification/diagnosis of a skin/tissue ailment without human intervention, that is, other than the patient being diagnosed. In other words, a doctor is not required to operate the present systems and methods.

Some embodiments of the invention are therefore intended for use by a patient or anyone with a potential dermatological issue. Such use may provide information allowing the patient to decide if he or she should seek help from a healthcare provider. In some embodiments, treatment information can be provided by the system.

The term “inquirer” as defined herein relates to a dermatologist, a physician, a researcher, a patient, a customer, a student or a software application.

Embodiments of the present invention include an installable software application (an “app”), installable on a device such as a computer or cellular telephone or the like. The software application can be provided in several languages and is designed to acquire and to analyze one or more digital images (photographs) of skin lesions from skin and/or various tissue types, to produce a differential diagnosis using one or more of the following: machine learning, image processing (analysis) technologies; and a questionnaire based on user input to narrow the differential diagnosis.

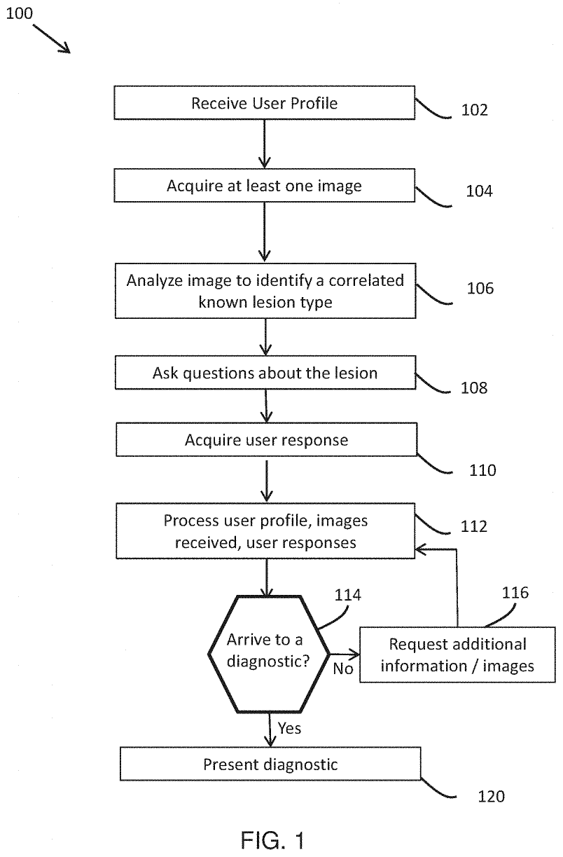

Reference is now made to FIG. 1 showing an embodiment of a skin-lesion diagnosis process. In step 100 the process starts when a user (also referred to herein interchangeably as “patient”) logs into an application which can be on any user device or terminal (computer, mobile phone, tablet etc.) in order to diagnose a skin lesion. In step 102 the system receives profile information about the user from one or more sources, internal and/or external to the system. Next, as will be discussed in detail below, the user provides one or more images of the lesion in the system analyzes the image or images in step 106 to identify a correlated known lesion type. Next, in step 108 the system provides the user with a questionnaire, and receives the user responses in step 110. In some alternative embodiments, the questionnaire may be provided before the images (steps 108, 110 before step 104).

The invention implements an algorithm (which may be used interchangeably herein with the term “program” or “process” or “method” or “system”) configured to use information from one or more questionnaires; and one or more digital images, to objectively define skin lesion properties. Moreover, a Desktop/Mobile application can use this algorithm, which will automatically improve diagnosis, specificity through machine learning, based on a pre-generated set of images and newly added images upon continued use of the present system itself and the (continually updated) existing published skin lesions databases. Alternatively, or additionally, the system may rely on available image processing techniques, which includes image comparison to standardized existing lesion databases and image recognition of specific areas/patterns.

The system acquires user profile from one or more available sources such as user profile databases, health organization databases, social networks etc. In addition, or instead, user profile information can be provided by the user itself, via questionnaires and/or previously received information. User profile can include information such as age, gender, existing medical conditions etc._{[GB1]}

At least one digital image _{[GB2]} of the inquirer's (patient's) lesion is received as input (step 104). The input can be generated through one or more imaging sources in a raw image stream, video or a still image/photo, and/or input from a digital image file and/or input from digital or printed databases and/or any other imaging source (local, remote, online, offline). A digital image may by in 2D or 3D in either a stills image or a video sequence. The system verifies that the image is acceptable to use in terms of quality, fuzziness, sharpness, resolution etc.

In some embodiments, the system may provide a physician-like interface such as a chatbot/dynamic questionnaire, operating as an app on a mobile client (such as a mobile personal computer, smartphone or custom processor-based camera device), which communicates with the patient, providing explanations such as how to take a picture (for example, with a mobile phone camera) of the lesion and reacting to questions that the user asks.

Initially, the method acquires one or more images (step 104) and acquires responses (step 110) to one or more questions asked to the user (step 108). Some information may be available through user profiles received in step 102.

Then the method analyzes the responses and the results of image analysis in step 112. If necessary (the system views in step 114 that it cannot reach a diagnostic) the method may ask the user more questions and/or instruct him to provide additional images in step 116. The system will then try again (in step 114) to reach a diagnostic.

In some embodiments, the questions are formulated, and the responses are processed by a natural language processing component [NLPC]. The natural language processing component can extract the information needed for diagnostics (disease features/characteristics) by information exchange between the user and NLPC. In addition, the NLPC can extract the information by analyzing any text, such as email, web page, chat interaction (chatbot). For example, if the user sent an email to his doctor explaining his condition, the system will extract information about the condition from such an email.

When the method has all the necessary information to reach a diagnostic, the method presents the diagnostic to the patient in step 120. The method may also recommend a treatment and/or provide useful information to the patient and/or perform management/follow-up (not shown).

The mobile client app reacts better and more precisely overtime, based on a growing knowledge base on the server side. The app may store the history of the patient or access it remotely which facilitates more sophisticated diagnosis. The system may store one or more labeled image of each condition. Every use of the system and additional images received helps the system, in a deep learning process, improve its diagnosis for that condition.

In some embodiments, a device such as a digital camera, which may be part of the mobile client or a peripheral can also be used with a lens with a magnifier comparable to a dermatoscope, allowing magnification for imaging small lesions or lesions emerging from the sub-epithelial. An illumination device (e.g., a lamp) can also be included to provide light with different wavelengths, such as ultraviolet light or infrared light.

### Methodology

### Questionnaire

The user (patient) is guided to follow a dynamic/adaptive questionnaire (dynamic/adaptive meaning that the set of questions changes based on the user's profile and the user's answers to previous questions and analysis of images provided).

Each answer to a specific question may affect subsequent questions presented to the user. Some examples are: if the user has indicated his age group to be younger than 12 years of age or that he is a male, then there will be no questions about pregnancy or other parameters specific to adults or women. The system may ask about shape/color change only if the user selects single lesion in a quantity question, and/or the system deduces it from image analysis. Shape/color change in its turn can point to a cancer possibility and will lead to other questions and recommendations.

If in condition region selection, the user selects eyes/eyelids/lips/fingers, the system may ask about potential swellings, since those areas are known to be prone to swellings.

More compacted rules are the algorithm decision tree itself. It's based on a set of features per disease. Once specific set exists, it indicates about specific disease.

In addition, each answer to the question minimizes the set of the potential diseases to be “analyzed” for the final diagnosis and vice versa, each “finding” by the computer vision/deep learning components will limit the number of question needed to be asked (e.g. If the computer vision component finds the lesion to be red there is no need to look for this information by a question to the user).

The content of the questions, wording, jargon, style, and/or UI/UEX of the app etc. may be adapted according to user of the system: a dermatologist, a physician, a pharmacist, cosmetics personnel, layman person etc_{[GB5]}. For example, physicians have good medical knowledge and want to spend as little time as possible. The same information may be presented to a physician on one or two screens, using the exact medical terms without explanations, allowing the physician to enter the right information in the fastest way, for example, entering keywords, free text, free text search etc. The patient, for example, may have more time to spend on the diagnosis process, so he will see more screens where the terms may be different, and more information will be provided about what the patient needs to enter. For example, for vesicle information, a patient may see a question such as “Do you a liquid/air like balloon on your skin?” (optionally with a visual example alongside) while the physician version may simply ask “Vesicle? Yes/No”.

without explanation

The system (application/process/chatbot) aims to change/improve the questions as more information is gathered and processed. For example, if discovered that the user age is 7, then the system will not ask questions about pregnancy state. With each additional information, the list of possible diagnostics results, can be reduced in many cases. Each fact about the disease and user conditions minimizes the list of possible diagnoses.

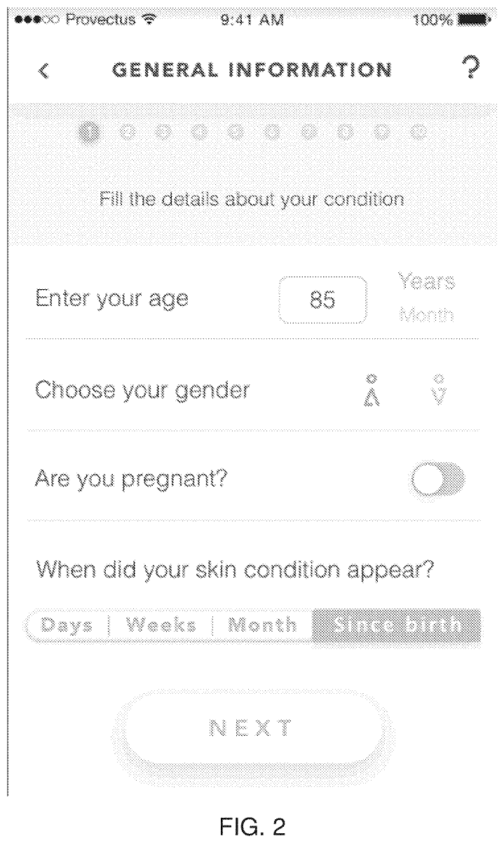

FIGS. 2-9 show examples of screenshots of a mobile application for a questionnaire according to the invention. In the example shown in FIG. 2 the user is requested to enter general information such as age, gender, pregnancy details and appearance of skin condition. Some information may already be available to the system, for example, through user profile information (age, gender . . . ) and thus may not be asked again.

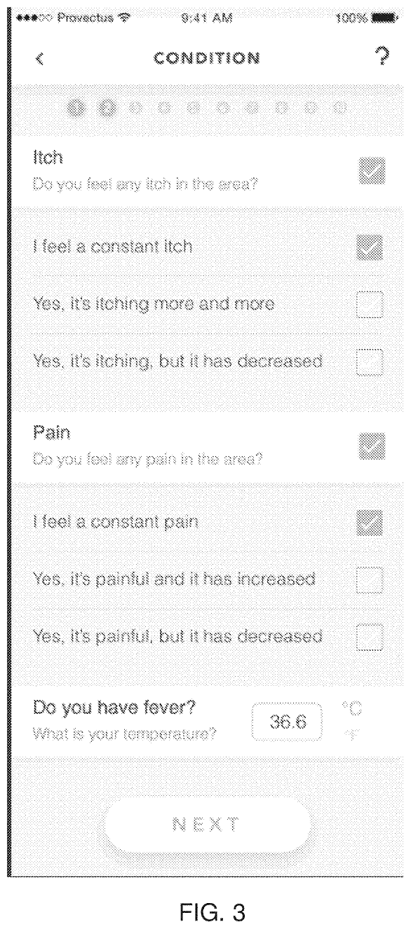

In the example shown in FIG. 3 the user is requested to enter specific information about the lesion such as itching information, pain information and fever.

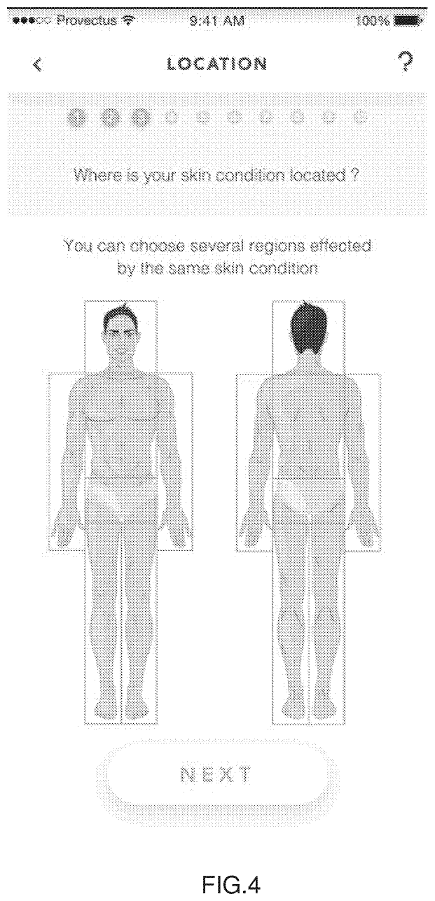

In the example shown in FIG. 4 the user is asked to provide general information (body part selection) where the lesion is located on his body.

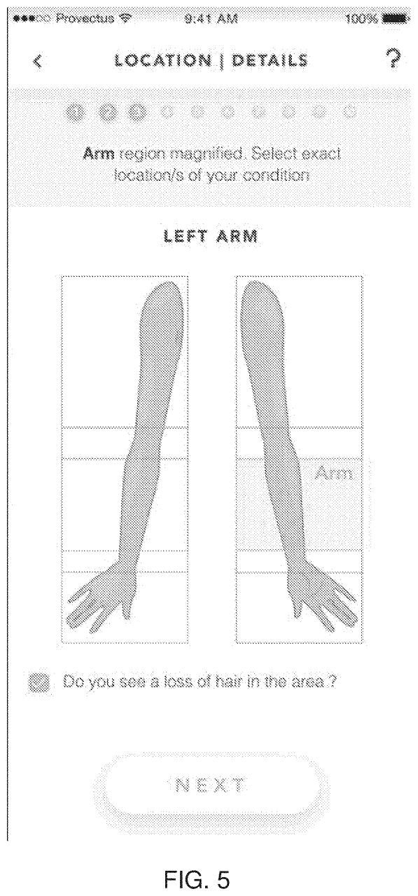

In the example shown in FIG. 5 the system shows the user the body part chosen in FIG. 4 and invites the user to show the specific area on the selected body part where the lesion is. For example, the user may first select the left arm (FIG. 4) and then select an area by the elbow of the left arm (FIG. 5).

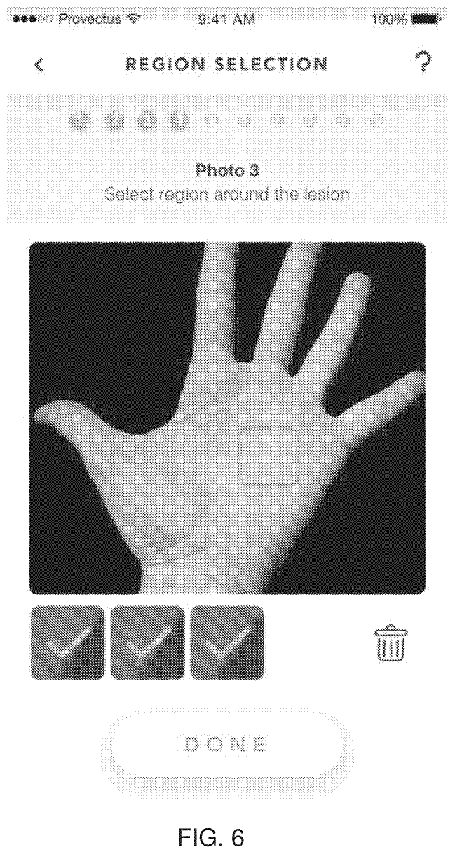

In the example shown in FIG. 6 the user is requested to focus on the actual region around the lesion.

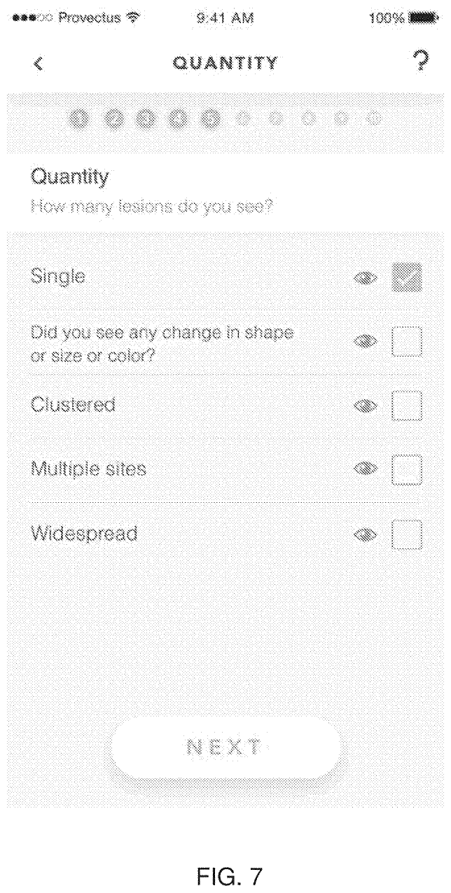

In the example shown in FIG. 7 the user is requested to enter information about the quantity of lesions he sees. Is it a single lesion or multiple lesions, are they clustered, multiple locations etc.

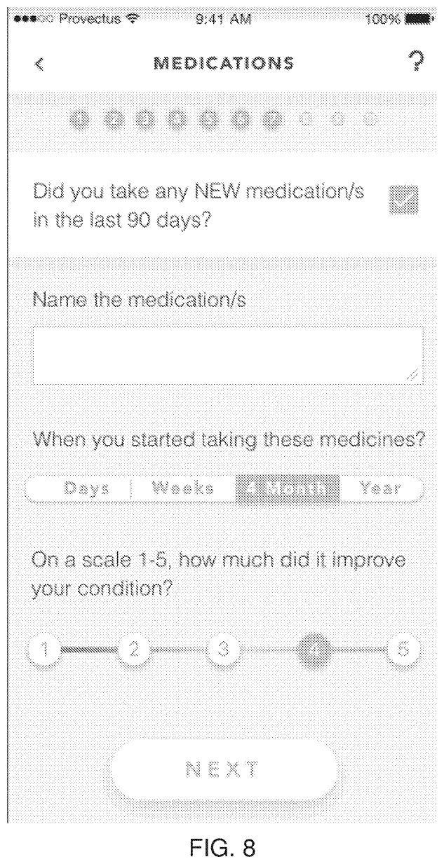

In the example shown in FIG. 8 the user is requested to enter information about any new medications he has started to take recently.

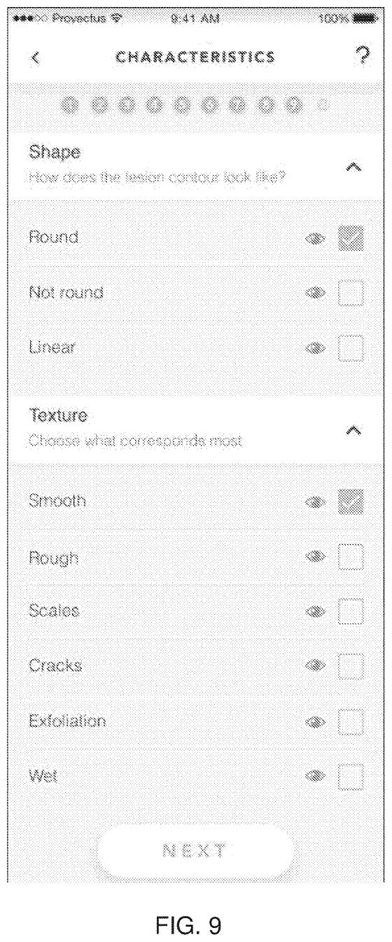

In the example shown in FIG. 9 the user is requested to enter information about the shape and texture of the lesion.

### Additional Information

In some embodiments, additional information about the patient can be obtained by analysing external sources such as social networks, web profiles, user-generated content like blogs, video channels etc. The application can apply analytical means such behavioural insights based on analysis of web behaviour, web profiles, etc. For example, if the user spent a lot of time in gambling websites, the system may consider high likelihood of increased stress levels.

In some embodiments, emotional information about the patient is extracted from external resources such as emotional state, mood. For example, if the patient just changed his profile status from “in relationship with . . . ” to “divorced”, the system may assume a fragile emotional state.

In some embodiments, environmental information about the patient is gathered from external sources relating to the surroundings of the patient, such as humidity levels, barometric pressure, pollution levels, temperature, sunshine, ultraviolet (UV) radiation levels etc. For example, if the patient is located in a dry area, the system may conclude about the effects on user skin condition; if the patient is located in Australia, there's a high likelihood of exposure to high UV radiation levels.

### Imaging Input

In addition to the questionnaire, the user is asked (before and/or after the questionnaire) to provide and/or take photos and/or raw video of the area of issue, and in some embodiments, any digital photo/video format is acceptable, including 3D images from any source (3D images using one or more lenses), including images taken under special conditions, that differ from standard white light, such as: ultraviolet/infrared or a dermatoscope generated image. Alternation of light conditions and/or type of the camera, allows the detection of lesions not visible in usual circumstances, such as detection of lesions under the skin. Additionally, any other tool can be added to access the diagnosed area.

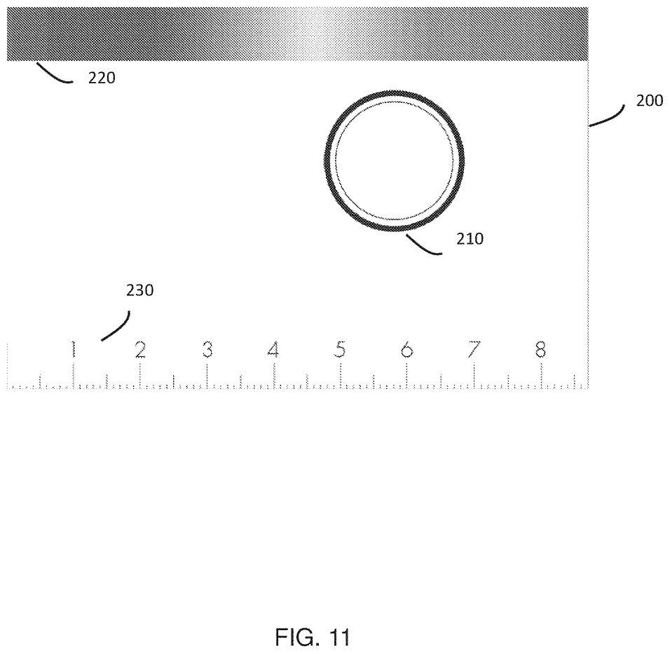

In some embodiments, image standardization (in terms of size/color/light saturation/elevation) can be achieved by using a predetermined reference printed size/color card (can be a business card-like device) as shown in FIG. 11. The reference card 200 shown in FIG. 11 has an opening 210 to surround (focus) the lesion, a color scale 220 so the lesion color can be identified by comparing its colors to the known color scale. The reference card 200 also has a length scale 230, for example, in centimeters or inches, so the true size of the lesion can be estimated from the image.

Alternatively, the system may use other standardization techniques such as a projected card (on the skin or any other surface) illuminated on the skin or white area near the skin by external or internal source of illumination.

Other options may include, for size normalization, asking the user to a known object near the lesion, for example, a smart phone, a coin, a bill or any other known object so the system can analyze the known object and by comparing the lesion dimension in the image to the known object dimensions in the image, derive/calculate the size of the lesion.

For color normalization, the user may be asked to place a known object (like a bill) or white paper (or white object) by the lesion, so the true color of the lesion can be estimated from the image by comparison to the object with the known color.

For light normalization, the user may be asked to illuminate the lesion with the light source of his mobile phone after providing the make and model of mobile phone used, and thus by the system can analyze the lesion in view of known lighting conditions.

Elevation normalization or data may be obtained by asking the user to place a known object such as a coin by the lesion, or by using advanced camera capabilities to provide elevation data, such as mobile phones with two frontal cameras that can take two photos (stereoscopic imaging like the human eyes) from different angles and thus provide 3-dimensional information (MESH) about the image.

### Processing

Identification of the nature of the skin lesions might be determinable via the questionnaire alone, but it is preferable to enhance identification accuracy via computer vision/deep learning components processing. By both means (steps), the lesion is initially defined and categorized in terms of shape, contour, size, whether it is raised or not and color and color homogeneity. The application software processes the digital pictures of the skin and/or other tissue and uses characteristics received through processing the image to provide characteristics, on which the diagnosis is based. In the application algorithm, lesions are categorized into at least, but not limited to, primary lesion and secondary lesions, as discussed immediately below.

Specific app algorithms use preset graphical rules to aid the questionnaire data to define whether the imaged lesion is a primary lesion such as: a macule, a papule, a nodule, plaque, a vesicle, bullae, a pustule, an ulcer, fissure atrophy or a secondary lesion such as: scales, crust/eschar, atrophy (which can be a primary lesion as well), lichenification or erosions, an excoriation, or a scar or keloids.

There are many techniques to define lesion properties through image analysis only (using deep learning). For example, color histogram related techniques can be used to define lesion color and peri-lesional area color (colors around the lesion area).

Lesion features are determined by extraction of characteristics from the image, such as lesion color, lesion elevation, cracks, bleeding, etc., using latest computer vision techniques, and DL using CNN. Pre-stored and/or pre-analyzed images were/are used to train the DL components, as well as definite answers received from the user through questionnaire. If the feature(s) match certain characteristic criteria that are definite to make a specific diagnosis by the algorithm, diagnosis, anamnesis, treatment summary is generated After the result data have been obtained, the algorithm determines and indicates the data as either definitive or as requiring further evaluation. If further data are required, the algorithm produces an additional questionnaire or requests that additional images be taken, which could be requested to be done immediately or in a time lapse (to define an evolving process or to define treatment effect). An algorithm decision will be made whether to use the help of human specialist dermatologist working with the application. In such cases, in some embodiments, the system can indicate if compensation for any help from a specialist is transferred directly or with any other available rewards option or with any other agreement with an Health Management Organization (HMO). In some embodiments, the algorithm may offer the inquirer to get a specialist opinion and connect the inquirer to a specialist

In parallel, further sub-definition of the skin lesion, in terms of dispersion is generated after processing of the initial digital image; and/or one or more additional images are requested; and/or the inquirers queried to respond to an additional questionnaire. The skin lesion sub-definition may be defined by distribution (such as a rash that is localized to a specific dermatome), a specific feature such as being an isolated lesion, clustered, confluent, morbilliform, spider angioma, scarlatiniform or universal, localized to a specific dermatome, satellite lesion (e.g. diaper candidiasis), involves hair follicles (follicular), along a site of injury (Koebner Phenomenon), serpiginous or reticular lesions.

The inquirer can be a health care provider such as a physician; or a researcher, a patient, a customer or an automatic machine.

The application is further configured to instruct the inquirer to photograph the lesion from different distances and/or locations/angles and/or different lighting and/or to slowly move above lesion or abnormality according to application instructions, which may be in a real time mode.

Images can be analyzed at multiple time points for characteristics such as change in growth, color, sharpness and so on based on multiple photographs over time and compared to each other.

The application may remind the inquirer to retake an image from the same distance after e.g. 3 months or at any other time and analyze the changes and alert/update the inquirer if there is reason to do so.

If a lesion was manipulated by a physician e.g. in a surgery, the application/program will take the operation time into account at time zero and analyze if the wound is going to heal on predicted time (e.g. as in stages of healing for a bedsore).

The program will request patient history (e.g. diabetes mellitus) which may lead to a slow wound healing process, and these details will be taken into account by the program.

The app may also define abnormalities and other lesions that may be not only from the skin such as lesions in the eyes, abnormal nose and ear anatomy, abnormal teeth (e.g. the notches in the biting surface, which can be symptoms/indications of congenital Syphilis). This lesions and abnormalities can be detected by the image processing, analyzed using the application's algorithm, which includes machine learning and a decision tree, and provide specific diagnosis and or recommendations.

Whenever a skin lesion is mentioned herein, it is intended to include other dermal or tissue abnormalities. Example of other tissues and abnormalities includes sclera, pupil, cornea, iris, parts of the nose, at least but not limited nasal septum, alar cartilage, nasal bone, conchae nasales, area of mouth (upper and lower lip, papilla incisive, superior and inferior frenulum of lip, uvula, palatine tonsil, glosso-palatine and pharyngopalatine arch, soft and hard and transverse palate, tongue, gingiva, teeth, lingual frenulum, salivary ducts (sublingual and submandibular), finger nails, hair, eyebrows, eyelashes, external auditory channels, urethra, testicles (e.g. hydrocele), nipples, blood vessels (e.g. characteristics of veins such as varicocele, caput medusae, stasis dermatitis, thrombosis e.g. in the leg; of arteria such as a hemorrhoid), lesion and abnormalities of the vagina (clitoris, labia majora and minora, urethra, skene's gland opening, tissues examined in a colposcopy—a procedure to examine a cervix, vagina and vulva for signs of disease; hymen, vaginal opening, Bartholin glands opening), penis (penis shaft, penis glans, urethra opening), dermatoscopic or histologic skin lesions.

Picture quality can be assessed initially by comparing it to a reference card and later by using standard means and accordingly the strength/weight of the data generated from the pictures is determinate. If picture quality is not be sufficient to make a reliable diagnosis, the inquirer is asked to repeat the picture taking and/or moving the camera in a video format above the skin and/or will be asked more questions. In some cases, the application determines that the pictures/data at hand is insufficient to make a reliable diagnosis and the inquirer is referred to a physician.

### Preparation of Initial Diagnosis Likelihood

The application algorithm uses a predefined set of rules applied to a digital image to objectively define skin lesion properties. Subtypes of a certain diagnosis are stored in a database to be shown later to the inquirer (either a patient; physician or another dermatology expert) to produce fine tuning in precise diagnosis (e.g. melanotic vs non-melanotic melanoma).

Likelihoods of differential diagnosis can be given in percentage, ranking, graphical means or a simpler way accordingly to the inquirer.

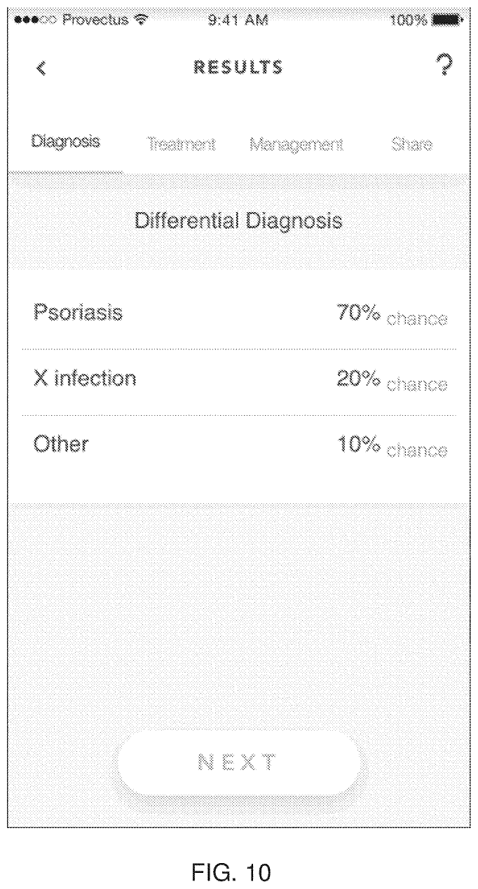

In the example shown in FIG. 10 the provides the diagnosis in terms of percentages. In the example shown, the likelihood is 70% Psoriasis, 20% X infection and 10% other.

### System for Fine Tuning of Final Diagnoses

The inquirer may be asked questions and may be shown one or more pictures of another lesion with the same diagnosis and/or other possible lesion types to further limit differential diagnosis.

In case further questions are needed, the questions will include query the patient regarding symptoms and general information like age, gender, ethnicity, area of residence and length of time spent at that residence, timing of appearance of the lesion, whether the lesion is permanent or comes and goes, changes and rate of change of the lesion and abnormal growth, as well as color and texture (e.g. flat, rough or raised bumps) of the lesion and central core (e.g. necrosis or a central dent/depression) and in comparison to the skin around it (e.g. does it look like a goose bump), is it glossy or waxy appearance, bump, wart like, scaly or crusty surface, does it have yellow crust, pain, tingling or numbness, itching, a burning feeling, previous exposure to sun, heat, cold or external material, insect bite, bleeding or easily bruised lesion, area of distribution (e.g. oral lesion, appearance of their tongues, neck, breast/chest, groin, eyelids, flexures or extensor areas, nails, hair, eyebrows, eyelashes, scrotum, nipples) and/or if there were previous episodes of the same lesion (e.g. hydrocele, zoster, herpes labialis, herpes in genital region, drug eruption, erythema multiforme, lymphomatoid papulosis).

The inquirer is asked about lesion properties including but not limited to raised, stiffness, softness, edema, is it fluid or air-filled lesion, change of color around lesion, crust. If relevant the inquirer might be asked about recent sexual intercourse and number of partners; systemic involvement previous or current fever, throat pain, joint or limb pain, hives, lymph node enlargement, darker or lighter area of the skin (e.g. café-au-lait spots), involvement of mucous membrane, anemia, family history, abnormal lab test (e.g. protein, electrophoresis, ESR), back pain, loss of weight, night sweats, bone pain, cough, shortness of breath, immunodeficiency, organ transplant and family history.

The guided questionnaire may ask all in the database defined questions and/or specific guided questions, concerning results from the image analysis process.

The inquirer, especially if it is a physician could be asked to enter all available data about the patient, such as vital signs (e.g. temperature, heart rate, respiratory rate), clinical presentation (e.g. mental status, septic shock, chills), blood gas analysis (e.g. pH, PO₂, PCO₂, HCO₃, lactate, base excess) biochemistry (e.g. Na⁺, K⁺, glucose, albumin, urea, creatinine, AST, ALT, CRP, total bilirubin, direct bilirubin, alkaline phosphatase), hematology (WBC, hemoglobin, hematocrit, neutrophils, platelets; anemia) and special dermatological signs such as: Nikolsky Sign, Auspitz sign, Bulla Spread Sign, Darier Sign, Hutchinson signs, Berlin's Sign as well as special laboratory tests such as: direct immunofluorescence, indirect immunofluorescence, ELISA, fungal smear and culture, Leishmaniasis Smear and culture.

Furthermore, the inquirer may be asked to enter information from patient background which will include history of cancer, recent travel, substance abuse, diabetes, immunosuppression, smoking, heart failure, previous infections (e.g. HIV, Hep A, Hep B, Influenzae, pseudomonas, syphilis, gonorrhea, chlamydia), surgeries, allergies, antibiotic or any other drug treatment, illnesses in the upper respiratory tract (e.g. enlarged tonsils, rhinorrhea, sore throat), illnesses in the lower respiratory tract (cough, chest pain, dyspnea, aspiration) illnesses in the abdomen region (e.g. Ascites, Crohn's disease, Cirrhosis), gastrointestinal and abdominal illnesses (e.g. diarrhea, vomiting, nausea, Splenomegaly, Peritoneal signs illnesses in the urinary tract (e.g. questions about incontinence, proteinuria, dysuria, flank pain), results from diagnostic apparatus (Ultrasound, CT-scan, X-ray, MRI, Pet-scan), results from examination (e.g. enlarged lymph nodes, auscultatory findings, percussion, reflexes), bone infections, illnesses of the central nervous system (e.g. Meningeal signs, headache) or any other question.

The inquirer such as a physician, a dermatology expert or a patient has the possibility to briefly summarize main complaints and thoughts in a written way or record them, so this information can be analyzed and also considered.

Details about person's origin allows the program to give higher priority to skin lesions common in a specific country.

Depending on who is using the App (patient or physician) the questionnaire may ask different or same questions, in a professional or a simple way, including explanations of the asked question (e.g. a question about hemoglobin may be asked in different ways such as: the application could query the physician if there is an anemia and/or request entry of blood results; the patient could be asked if he felt weak in the last couple of weeks, as weakness could be an indirect sign of anemia. Also, the application UI/UEX will be uniquely tailored to the user needs. Primary physicians may receive different application behavior and inputs.

### Confirmation of Final Differential Diagnosis

The likelihood of a differential diagnosis is generated by the algorithm and influenced by the data gathering from, but not necessarily limited to, (a) detailed questionnaires based on decision tree, (b) automatic visual image processing using the specific indicators identifying each skin lesion, and (c) the deep learning process using datasets—either self-constructed or from labeled skin lesions available in public domains. In some embodiments, the resulting diagnosis may be presented as a percentage of likelihood (of a particular diagnosis), graphical means, or ranking and linked to a set of previously obtained skin lesion digital images. The likelihood of a certain diagnose for each step or question may be presented to the inquirer so one can understand how the data gathering increases the likelihood of a specific diagnosis.

If needed, the inquirer will be asked to compare an initial image(s) to similar pre-stored and self-generated data base pictures in order to strengthen accuracy. When in doubt in regard to leading differential diagnosis, further investigation may be suggested, and a second opinion may be obtained. An expert opinion option will be available in two forms. The first asking the patient whether to schedule an appointment with a field expert. The second is a fast consultation option in which probable diagnosis, differential diagnosis and the data leading to that diagnosis will be presented directly to a dermatologist via web site or via a telemedical support service, giving the patient an easy and fast option to ratify/confirm the technology diagnosis and management plan.

### Rule Out Investigation for Malignant Lesion

A specific emphasis might be on either ruling out a malignant lesion or setting an alert for immediate investigation.

Digital image processing as well as the questionnaire might reveal specific elements such as asymmetry, irregular, blurred or ragged borders, irregular or different colors or darkness within one lesion (e.g. dark speckles inside), large diameter, or change in shape, color or size among the presence of scaly crust, ulcer, slow or lack of healing, bleeding, purple patches, specific area of distribution (e.g. anus, genitalia, or eyelid nodules). Every image is tested for excluding criteria—if one of the five criteria is TRUE, then the system does NOT auto-diagnose, but alerts the patient to go see the closest dermatologist:

1. Solitary pigmented lesion (Specifically dark colors: black, blue, deep brown or combinations of these colors)
2. Bleeding lesion solitary or multiple
3. Long standing solitary lesion (more than one month) not responding to treatment
4. Long standing solitary lesion which changed by: color, size, contour, started to itch or to hurt, the skin around the lesion changed by color
5. Any kind of eruption solitary, few lesions or wide spread eruption, not responding in two weeks of recommended treatment, OTC or treatment requires prescription

If a pre-cancerous situation (such as at least but not limited Actinic keratosis) was found, the app will remind the patient at different times to see a physician and/or the patient's physician to request a follow up appointment and/or repeat the image recognition process to check if changes occurred and calculate the risk of getting a skin cancer and/or providing updated information about the pre-cancer.

The app can be configured to instruct the inquirer to adjust the camera and use specific filters or to zoom in on the lesion if it is required, especially to rule out a malignancy.

### Suggested Further Investigation

The computer algorithm will define if additional investigation is needed by means of (but not limited to) additional digital images, lab procedures such as blood work, serology, urinalysis, imaging (e.g. x-rays, US), physical exam and biopsies.

Upon a specific dilemma, a flag may be raised to determine the need for an expert review of data.

### Self-Generated Data Base Acquired Through Machine Learning Techniques

Machine learning techniques are able to develop implicit or explicit models that categorize patterns in a data set. Machine learning algorithms for categorizing lesions may include classification, or “clustering” algorithms, including Bayesian networks and Markov models, and neural networks, in particular convolutional neural networks (CNNs). In an embodiment of the present invention, the training set may be generated by a supervised machine learning algorithm that merges features of lesion images from a database of images together with descriptive/symptomatic features provided by medical professionals. The process may also include generating probabilities of identification based on descriptive features and on the actual rate of appearance of certain types of patterns during the training process. The database may also include thresholds of recognition that delimit features of lesions.

Once a new digital image diagnosis is confirmed, e.g. by a dermatologist or histology (the results of pathology and/or biopsy) it will be added to the image database. The app will use an algorithm which will improve diagnosis specificity automatically through comparing the new processed image to pre-saved digital images of the same making future analysis both faster and more accurate. The accumulated big data will/may be revised by an expert dermatologist in order to improve the algorithm's accuracy. Big data acquired through machine learning typically not only grows faster than human experience but is also not limited in terms of memory or recall errors, or subjectively biased as human beings are.

The self-generated data base will be processed by an application algorithm that can use a set of digital graphic rules and properties to objectively define correct skin lesion diagnoses.

A comparison of the dermatologist or histology diagnosis to the differential diagnosis will enhance accuracy of likelihood in future inquiring.

### Algorithms

Many different algorithms may lead to the diagnosis. One algorithm can be enough or the combination of many will be performed and joined. Examples are using many algorithms of ‘same type’ such as algorithm used to identify specific visual indicators such as color/size/irregularity/shape; or using algorithm of ‘different types’ such as algorithm to identify visual indicators together with an algorithm to define skin lesion types by a guided questionnaire and/or algorithm using machine learning techniques.

These options of operations can be extended to include the following features:

One specific algorithm or/and a color chart is used to normalize the colors of the camera and skin lesion colors. For example, an algorithm could provide to every color a specific number.

Another algorithm recognizes the form of the lesion by defining the borders of the lesion or/and comparing them to forms of already validated skin lesion.

Another algorithm separates the image in different parts and analyze each part itself and compare later different parts with each other. For example, the algorithm may analyze the intra-relationship of one lesion (by analyzing at least but not limited the distribution, color, shape) and defining groups that represent specific criteria. A group will have at least one characteristic in common (e.g. color, sharpness or shape) that another group doesn't have. At least one of these criteria will be more frequently presented (e.g. color) then another one (e.g. shape).

Another algorithm identifies secondary lesions or allergic phenomena by certain questions such as about previous disease or itching.

Another algorithm uses deep learning techniques to recognize another ‘lesion of the same type’ in comparison to stored pictures/images located in a database of the same kind of lesion.

Part of the process could be to put parts of the image affected by the lesion in one group and to exclude parts that are not affected from the image or put them into another group. Image groups (representing different parts of the same lesions) can be analyzed by different characteristics (e.g. distribution, color, shape and sharpness). These characteristics could get different priorities, values and/or be analyzed in another way.

In some cases, the color and distribution of the color, on a specific skin region can be a more important characteristic than the lesion's shape.

For example, for a “skin-rash”: The criteria “color” might get a very high diagnosis “analysis weighting value or factor” and the “shape” would receive a high, but slightly lower diagnosis “analysis weighting value or factor” in the analyzing process.

In another example, Pityriasis versicolor, the criteria “shape” would receive a high diagnosis “weighting value or factor”, because there is no dynamic color change in the lesion, compared to a skin-rash. In such a case, the program analyzes the shape as the most important criterion although the program also analyzes the color of the lesion as an important secondary clue to the diagnosis.

Another algorithm may also analyze the whole picture as one image, comparing the image to other skin lesions in database using deep learning techniques.

Another algorithm could compare the skin lesion with normal skin to determine at least but not limited the distribution, color, shape and sharpness of the skin lesion. It could also compare the skin lesion with normal skin, to clarify the stage of a skin condition. E.g. wounds can be compared with each other overtime, to predict the healing process and stage (e.g. bedsore).

Another algorithm may try to match previous lesions with new lesions, in order to define points that are specific and occurs in every lesion of the same disease.

Another algorithm may try to create stencils, that can be matched to a new lesion, to provide a diagnose or to show in percentage how exact the match is.

A “healthy” image of every body part may serve as a template for later analysis.

A “lesion” may serve as a template for later analysis at least but not limited to analyze if the lesion responds to treatment. That information is then added to the imaging database.

A user may be asked to mark the lesion on the phone or any other video/image device to define the borders of the lesion in order to help the algorithm to detect a more accurate diagnosis.

The system algorithms can compare healthy areas and lesion areas to gain more information about the patient.

In some embodiments, the application is used to diagnose side effects after a drug treatment. The application applies the same questionnaire/image analysis process as described above in order to determine if a lesion is actually a side effect of the drug the user has been taking. Depending on the diagnosis results, the response of the system can be:
1. It's not a side effect of the drug, seek professional help;
2. It's a side effect of the drug, apply treatment “X”;
3. It's a side effect of the drug, stop using the drug immediately;
4. Take another picture (of another region and/or in some future time).

In some embodiments, the application is used to diagnose side-effects after a drug treatment, wherein the side-effects include side-effects that are not skin lesions.

The algorithm engine is a dynamic engine that can be constantly updated as new research, new regulations, new procedures, new treatments are available.

The system may also include a custom reference card shown in FIG. 10, which may provide aids such as a spectrum of colors, predetermined elevations and a ruler helping the app to calibrate the colors and the size of the lesion. The card-like device may include a hole, which may be round or have other forms, such that the hole can be placed on the skin lesion thus identifying the region of interest while also decreasing the background noise. A ruler may help the app to recognize the size of the lesion. The predetermined elevations help determine the size of the lesion by comparing to the shade created by the elevations.

### Features, Options and Embodiments of the Invention

1. Digital recognition of skin and tissue lesions and abnormalities.

2. Skin lesion or skin disease is identified by a set of guided questionnaires combined with image analysis of pictures of the lesion.

3. A self-generated database acquired through machine learning techniques.

4. Application software capable of giving fast diagnoses, available on devices such as a computer or mobile phone.

5. Application is designed to analyze a digital photograph of lesions from skin or/and all kinds of tissue types using both pre-stored data as well as self-generated characteristic image properties.

6. Algorithms can use a set of digital graphic rules to objectively define skin lesion properties.

7. Application uses algorithms to improve diagnosis specificity automatically through comparing a new processed image to pre-saved digital images of the same characteristic as well as to a self-generated database acquired through machine learning techniques.

8. Likelihood of the different optional diagnosis is provided.

9. A digital image of the inquirer's lesion is received as an input. The input can be generated through raw image stream, video or still image/photo, and/or input from a digital image file and/or input from digital or printed databases and/or any other source.

10. The image input is received from the inquirer who can be a physician, researcher, patient, customer or automatic machine.

11. The lesion image can be captured under normal white light or different other waves, such as ultraviolet or infrared light. Different light and wave conditions allow detection of lesions and features, not visible under white light, for example, identifying lesions under the skin.

12. Supporting different kind of cameras such as cellular phone cameras and/or cameras using ultraviolet light will record lesions.

13. The lesion image can be captured in 2D or 3D image sequences or via a video that can be processed by analyzing each individual frame of the video.

14. The inquirer can be asked to use lighting to obtain a brighter image (e.g. as might be required for an image of the inside of one's mouth) or any other imaging accessory that could help the inquirer or/and the camera to provide an in quality improved image.

15. Lesions of the skin and tissue such as nails and sclera can be detected by a digital image and initially defined and categorized by means of shape, contour, size, texture, color and color homogeneity.

16. Lesions can be categorized into primary lesions, secondary lesions, such as scratch, trauma (including deformations e.g. of the thorax) infections and distribution.

17. Specific application algorithms can use pre-set graphical rules to define whether the lesion is macule, papule, nodule, plaque, vesicle, bullae, pustules, eczematoid, telangiectasia, petechiae, ecchymoses, purpura, annular, wheal and flare, target lesions, guttate like lesions, linear lesion, multiform, and specifically suspected tumor.

18. Features such as scale, crust, atrophy, lichenification or erosions, excoriation, fissure, ulcer, scar or keloids and eschar can be defined in comparison to pre-stored images, and matched to certain characteristic criteria that are definite to make a specific diagnosis by the algorithm that is analyzing it.

19. After the result data have been obtained the data is marked as definitive; or further evaluation is initiated by the algorithm which involves a questionnaire and/or additional images of the lesions (different angle, close-up, any standardization technique etc.).

20. In parallel, further sub-definition of skin lesions in terms of dispersion can be generated after processing the initial digital image, and/or additional images are requested, and/or the inquirer is asked to fill a questionnaire.

21. The skin lesion may be defined as isolated lesion, clustered, confluent, morbilliform, spider angioma, scarlatiniform or universal, localized to a specific dermatome, satellite lesion (e.g. diaper candidiasis), involve hair follicles (follicular), along a site of injury (Koebner phenomenon), serpiginous or reticular lesions.

22. The inquirer can be asked either to take pictures of the lesion from different distances and locations or to slowly move the camera above the lesion or abnormality, according to the application's instructions, which may be in real time.

23. Imaging can be performed at multiple time periods and characteristics like growth, color, sharpness can be respectively compared over time.

24. The application may remind the inquirer to repeat an image from the same distance after e.g. 3 months or at any other time and will analyze the changes and alert the inquirer about them.

25. The application may output a message or an alarm to see a physician as fast as possible or inform the user about regular self-limiting properties of the lesion.

26. If a lesion was manipulated by a physician e.g. in a surgery, the application takes the date of the operation into account and analyzes if the wound is going to heal within a predicted time (e.g. stages of healing for bedsore).

27. The application can ask details from patient history (e.g. diabetes mellitus) which may lead to a slow wound healing process and these details will be taken into account by the program.

28. The system is capable of performing all of the other listed features, also with respect to tissue lesion or abnormalities such as sclera, pupil, cornea, iris, parts of the nose, nasal septum, alar cartilage, nasal bone, conchae nasales, area of mouth (upper and lower lip, Papilla incisiva, superior and inferior frenulum of lip, Uvula, Palatine tonsil, Glossopalatine and Pharyngopalatine arch, Soft and Hard and transverse palate, tongue, gingiva, teeth, lingual frenulum, salivary ducts (sublingual and submandibular)), finger nails, hair, eyebrow, eyelash, external auditory channel, urethra, testicles (e.g. hydrocele), nipple, blood vessels (characteristics of veins such as varicocele, caput medusae, stasis dermatitis, thrombosis e.g. in the leg; of arteria such as hemorrhoid), lesion and abnormalities of the vagina (Clitoris, Labia majora and minora, Urethra, Skene's gland opening, Hymen, Vaginal opening, Bartholin Glands opening), penis (penis shaft, penis glans, urethra opening), anus.

29. The system/method can analyze a digital image of a skin or tissue lesion or abnormality or pathology by means of image processing and comparison to a predefined set of characteristic properties.

30. Confirmation of final differential diagnosis may be aided by an additional questionnaire, and/or an expert opinion, and/or comparison of initial image to pre-stored lesion of the same.

31. A guided questionnaire can provide on collected data also a skin lesion independent diagnosis, depending at least but not limited on symptoms.

32. The system's algorithm(s) can use comparison of healthy areas and lesion areas.

33. This algorithm can use a “lesion” as a template for later analysis at least but not limited to analyze if the lesion responds to the treatment. add to database

34. This system/method is capable of setting appointments or offering contact information to geographically close located physician and hospitals.

35. This system/method is capable of providing specific information concerning skin conditions and can keep up-to-date for the latest research about specific skin conditions.

36. The application is capable of analyzing the recorded or/and written words by the user about conditions that have not been asked by the guided questionnaire.

37. The system/method can provide a differential diagnosis in percentage, ranking, graphical means or a simpler manner according to the inquirer.

38. A handy size reference card tool, such as a specific designed business card may be used to measure the size of the lesion, the color of the lesion and the form of the region of interest. This “tool” may be projected by a stand-alone device or a mobile app.

39. A connection can be made by GEO-location to the closest physician.

40. Patient history can be transmitted to the closest physician.

41. Treatment recommendations such as an OTC prescription can be advised.

42. Treatment recommendations such as Non-OTC (e.g. antibiotics) prescription can be advised.

43. An image analysis of a medicament for treating of skin lesions can be recognized by an image of the medicament packaging.

44. When a medicament is recognized by image analysis, information about proper usage can be provided.

45. The application is capable of merging/combining information received from the patient by method of questioning and information received by analyzing a photo of a condition/lesion.

46. The application is capable of giving follow up on specific skin conditions/lesions, such as chronic skin lesions.

47. The application can list nearest pharmacies based on GEO location (proximity and opening hours).

48. The application can provide information about availability of specific medicament in the listed pharmacy.

49. The platform can provide a direct purchase option for medicament in the listed pharmacy.

It will be readily apparent that the various methods and algorithms described herein may be implemented by, e.g., appropriately programmed general purpose computers and computing devices. Typically, a processor (e.g., one or more microprocessors) will receive instructions from a memory or like device, and execute those instructions, thereby performing one or more processes defined by those instructions. Further, programs that implement such methods and algorithms may be stored and transmitted using a variety of media in a number of manners. In some embodiments, hard-wired circuitry or custom hardware may be used in place of, or in combination with, software instructions for implementation of the processes of various embodiments. Thus, embodiments are not limited to any specific combination of hardware and software.

A “processor” means any one or more microprocessors, central processing units (CPUs), computing devices, microcontrollers, digital signal processors, or like devices.

The term “computer-readable medium” refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models and/or distributed databases) could be used to store and manipulate the data types described herein. Likewise, object methods or behaviors of a database can be used to implement various processes, such as the described herein. In addition, the databases may, in a known manner, be stored locally or remotely from a device which accesses data in such a database.

The present invention can be configured to work in a network environment including a computer that is in communication, via a communications network, with one or more devices. The computer may communicate with the devices directly or indirectly, via a wired or wireless medium such as the Internet, LAN, WAN or Ethernet, or via any appropriate communications means or combination of communications means. Each of the devices may comprise computers, such as those based on the Intel® Pentium® or Centrino™ processor, that are adapted to communicate with the computer. Any number and type of machines may be in communication with the computer.

1. A computer system for diagnosing skin lesions of a user, comprising:
a processor; and
a memory communicatively coupled to the processor comprising computer-readable instructions that when executed by the processor cause the computer system to:
i. acquire via the processor information about the user profile;
ii. acquire at least one user lesion image;
iii. apply via the processor one or more pattern recognition or deep learning techniques on the acquired image to identify a correlated known lesion type;
iv. present the user via the processor a first question about the lesion;
v. acquire via the processor the user's response to said first question;
vi. process via the processor the user's response, user lesion image and user profile and if a diagnostic cannot be made, decide whether to formulate another question to the user or request an additional image of the lesion;
vii. repeat steps ii to vi until a diagnostic can be made; and
viii. present the diagnostic to the user via the processor.

2. The system of claim 1, wherein the system comprises a mobile computing device, wherein the mobile computing device comprises a digital camera configured to acquire the patient lesion image, and wherein the mobile computing device is further configured with an application for acquiring the patient data.

3. The system of claim 1, wherein the questions are formulated and the responses processed by a natural language processing component [NLPC].

4. The system of claim 1, wherein additional information about the patient is gathered from external sources.

5. The system of claim 4, wherein emotional information about the patient is extracted from said external resources.

6. The system of claim 1, wherein environmental information about the patient is gathered from said external sources.

7. The system of claim 1, wherein said system is further adapted to acquire additional information comprising measuring skin subcutaneous data.

8. The system of claim 7, wherein said additional information is obtained by using ultrasound, UV light, infrared light or a photovoltaic cell_{[GB11]}.

9. The system of claim 1, wherein the system recommends a treatment along with the diagnosis.

10. An image recognition reference card, comprising:
at least one inner opening;
a ruler;
a color scale,
such that when a user places the inner opening above a skin lesion and takes a photograph of the lesion, the ruler and color scale help determine the actual color and size of the lesion.

11. The reference card of claim 10, wherein the opening is covered with a transparent material.

12. A computer system for managing dermatological side effects of a drug administered to a patient, comprising:
a processor; and
a memory communicatively coupled to the processor comprising computer-readable instructions that when executed by the processor cause the computer system to:
acquire via the processor at least one image of a suspected skin lesion;
acquire via the processor at least one response from the patient, for a question generated by the system;
analyze the at least one image use pattern recognition and deep learning techniques;
analyze the at least one response using an inference engine applying a dermatological rule-based decision tree; and
inform the user via the processor whether the skin lesion is related or not to the administered drug.

13. The system according to claim 12, wherein the system recommends an appropriate treatment if the skin lesion is identified to be related to the administered drug.

14. The system according to claim 12, wherein the administered drug is Vectibix (Panitumumab).
